(19) United States
(12) Patent Application Publication
(10) Pub. No.: US 2025/0205482 A1
(43) Pub. Date: Jun. 26, 2025

(54) DETERMINING TRANSDUCER LOCATIONS FOR DELIVERY OF TUMOR TREATING FIELDS USING SIMULATIONS BASED ON MODELS OF HEALTHY SUBJECTS

(71) Applicant: Novocure GmbH, Root D4 (CH)

(72) Inventors: Oren PELES ZEEVI, Haifa (IL); Amit FELDMAN, Haifa (IL)

(73) Assignee: Novocure GmbH, Root D4 (CH)

(21) Appl. No.: 18/961,009

(22) Filed: Nov. 26, 2024

Related U.S. Application Data
(60) Provisional application No. 63/613,835, filed on Dec. 22, 2023.

(51) Int. Cl. A61N 1/36; A61N 1/02; G06T 7/00; G16H 50/20

(52) U.S. Cl. CPC A61N 1/36002; A61N 1/025; A61N 1/36031; A61N 1/36034; G06T 7/0014; G16H 50/20; G06T 2207/10081; G06T 2207/10088; G06T 2207/10104; G06T 2207/20081; G06T 2207/20104; G06T 2207/30096

(57) ABSTRACT

A method for determining transducer locations for delivery of tumor treating fields based on models of healthy subjects, including: receiving a medical image of a subject having an abnormality; receiving a selection of a healthy model from a plurality of healthy models, the healthy model being representative of the subject, the selection based on the medical image of the subject; receiving a selection of locations on the healthy model to place transducers to treat the abnormality of the subject without identifying a location of the abnormality in the healthy model; receiving an indication of a region of interest in the healthy model; and calculating for each of the locations, a dosage of tumor treating fields treatment in the region of interest of the healthy model without modifying the healthy model to include abnormal tissue.

### CROSS-REFERENCE TO RELATED APPLICATIONS

This Application claims priority to U.S. Provisional Application No. 63/613,835, filed Dec. 22, 2023, which is incorporated by reference herein in its entirety.

This Application is related to U.S. Provisional Application No. 63/613,858, filed Dec. 22, 2023, which is incorporated by reference herein in its entirety.

### BACKGROUND

Tumor treating fields (TTFields) are low intensity alternating electric fields within the intermediate frequency range (for example, 50 kHz to 1 MHz), which may be used to treat tumors as described in U.S. Pat. No. 7,565,205. TTFields are induced non-invasively into a region of interest by transducers placed on the patient's body and applying alternating current (AC) voltages between the transducers. Conventionally, a first pair of transducers and a second pair of transducers are placed on the subject's body. AC voltage is applied between the first pair of transducers for a first interval of time to generate an electric field with field lines generally running in the front-back direction. Then, AC voltage is applied at the same frequency between the second pair of transducers for a second interval of time to generate an electric field with field lines generally running in the right-left direction. The system then repeats this two-step sequence throughout the treatment.

### BRIEF DESCRIPTION OF THE DRAWINGS

FIG. 1A depicts an example method for determining transducer locations for delivery of tumor treating fields using simulations based on models of healthy subjects according to an embodiment.

FIG. 1B depicts an example method for generating healthy models according to an embodiment.

FIG. 2 depicts examples of healthy models.

FIG. 3 depicts an example plot used for clustering healthy models.

FIG. 4 depicts an example plot used for clustering healthy models.

FIG. 5 depicts an example plot of clusters of healthy models.

FIG. 6 depicts an example apparatus to apply alternating electric fields to a subject's body.

FIGS. 7A-7B illustrate schematic views of exemplary design of a transducer for applying alternating electric fields.

FIG. 8 depicts an example placement of transducers on a subject's head.

FIG. 9 depicts an example computer apparatus.

Various embodiments are described in detail below with reference to the accompanying drawings, wherein like reference numerals represent like elements.

### DESCRIPTION OF EMBODIMENTS

When generating transducer layouts for application of tumor treating fields (TTFields) for a subject, medical images of the subject are often used. Examples of such medical images are magnetic resonance imaging (MRI) scans, computed tomography (CT) medical image, positron emission tomography (PET) medical images, and/or the like including combinations and/or multiples thereof. Segmentation is often performed on medical images to divide a medical image into two or more segments with each segment representing a different object of interest. A medical image is often segmented to extract or isolate an object of interest (e.g., abnormal tissue) in the medical image from other structures (e.g., healthy tissue). As discovered by the inventors, performing segmentation on a medical image of a subject uses significant resources in terms of computing power and healthcare provider time. For example, to segment an image, the image is sliced, and an abnormality is segmented from healthy tissue in each slice, requiring significant time from the healthcare provider and significant computing resources to perform the segmentation. Moreover, segmentation on medical images of certain regions of the body are particularly computationally demanding. For example, medical images of a head typically show about five different types of tissues, while medical images of a torso show about twenty different types of tissues, making segmenting medical images of a torso significantly more computationally demanding than segmenting medical images of a head. Medical images of a torso are also significantly larger than medical images of a torso, for example, due to the relative size different in heads and torsos. Consequently, it may take a few minutes to segment a medical image of a head but upwards of an hour to segment a medical image of a torso.

One or more embodiments described herein provide for generating transducer layouts for application of TTFields for a subject without performing segmentation. In some embodiments, a healthy model may be selected from multiple healthy models, the selected healthy model being selected to represent a subject. For example, a healthy model with similar characteristics/properties (e.g., height, weight, body mass, gender, etc.) to the subject may be selected. For example, measurements of the healthy subject are taken from the medical image and compared to corresponding measurements in healthy models. A healthy model is then selected based on the measurements. The healthy model is then used to select locations to place transducers to treat the abnormality. A dosage of TTFields treatment is then calculated, for each of the locations, for a region of interest in the healthy model of the subject without modifying the healthy model to include abnormal tissue. Thus, a subject-specific transducer layout is generated for the subject without segmenting medical images of the subject by using the healthy models.

The embodiments described herein further provide a practical application of generating transducer layouts by avoiding the need to perform segmentation on medical images of the subject. By using the healthy models, the tissue conductivity is considered when generating transducer layouts for treating the subject with TTFields without having to perform segmentation on medical images of the subject. This approach yields more effective TTFields treatment for the subject while saving computational processing resources and healthcare provider time. These and other technical improvements may be realized using the one or more embodiments described herein.

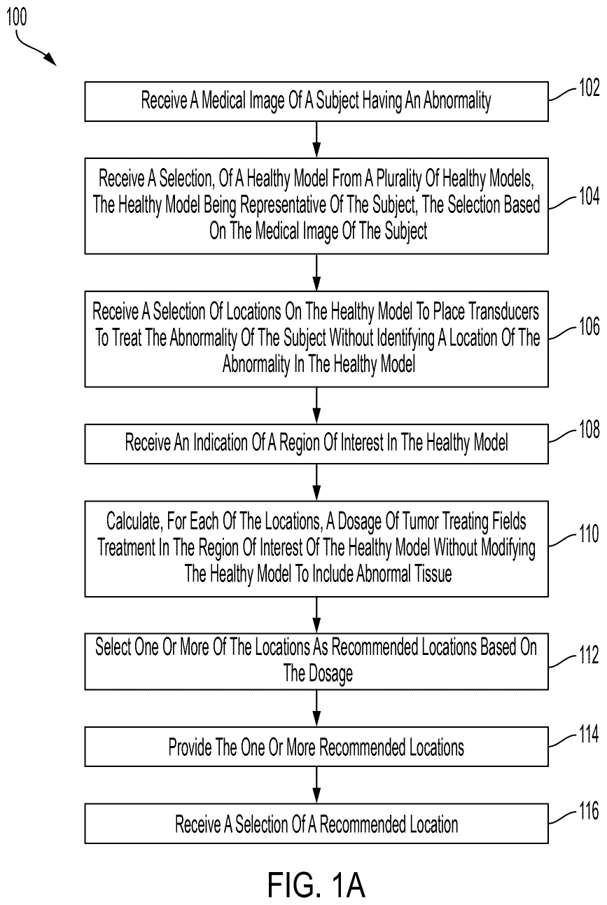

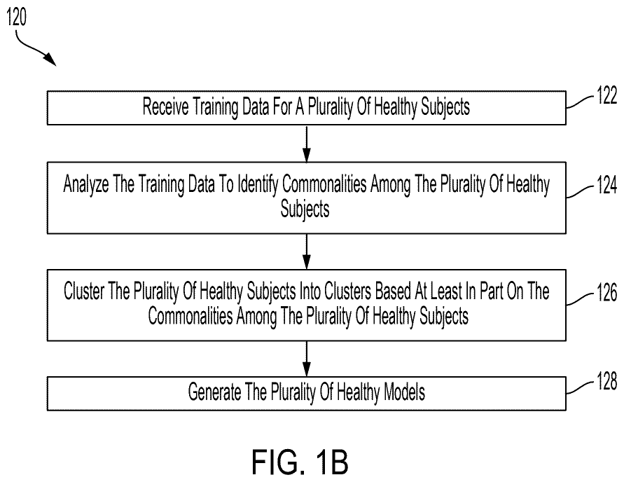

FIG. 1A depicts an example method for determining transducer locations for delivery of tumor treating fields using simulations based on models of healthy subjects. Certain steps of the method 100 are described as computer-implemented steps. The computer may be, for example, any device comprising one or more processors and memory accessible by the one or more processors, the memory storing instructions that when executed by the one or more processors cause the computer to perform the relevant steps of the method 100. The method 100 may be implemented by any suitable system or apparatus, such as the apparatus 900 of FIG. 9. While an order of operations is indicated in FIG. 1A for illustrative purposes, the timing and ordering of such operations may vary where appropriate without negating the purpose and advantages of the examples set forth in detail herein.

With reference to FIG. 1A, at step 102, the method 100 includes receiving a medical image of a subject having an abnormality. According to one or more embodiments described herein, the abnormality is a tumor or another type of non-healthy tissue. According to one or more embodiments described herein, the medical image is a computed tomography (CT) image, a magnetic resonance imaging (MRI) medical image, a positron emission tomography (PET) medical image, and/or the like including combinations and/or multiples thereof. The medical image can be of all or a portion of the subject. For example, the medical image can be an MRI, CT, or PET image of a head of the subject, a torso of the subject, and/or another portion of the subject.

At step 104, the method 100 includes receiving a selection, of a healthy model from a plurality of healthy models, the healthy model being representative of the subject, the selection based on the medical image of the subject. In some embodiments, healthy models may be models of subjects known to be without or substantially without abnormalities in an area of interest (e.g., a head, torso, and/or the like including combinations and/or multiples thereof). For example, medical images of healthy subjects (e.g., those known to be without abnormalities in an area of interest) are captured and used to create healthy models of the healthy subjects. According to one or more embodiments described herein, the healthy model is selected from the plurality of healthy models based at least in part on a medical image of the subject (e.g., MRI image(s), CT image(s), PET image(s)), a physical measurement of the subject (e.g., a circumference of the subject's head or torso), a classification of the subject (e.g., a gender of the subject), and/or the like including combinations and/or multiples thereof.

According to one or more embodiments described herein, the plurality of healthy models is based on healthy subjects clustered into groups. For example, the healthy models can be grouped based on characteristics of the healthy subjects from with the healthy models were created. The healthy models can be grouped according to any suitable characteristic(s), such as weight of the subject, height of the subject, body mass of the subject, gender of the subject, health information about the subject (e.g., previously surgical procedures, known diseases), and/or the like including combinations and/or multiples thereof. According to one or more embodiments described herein, each of the plurality of healthy models is representative of a group of healthy subjects without abnormalities. According to one or more embodiments described herein, each of the plurality of healthy models is segmented based on tissue type. The selection of the healthy model can be determined automatically (e.g., based on analyzing one or more medical images of the subject to identify similarities to one of the heathy models), manually (e.g., a healthcare provider can select a healthy model from the plurality of healthy models), and/or the like including combinations and/or multiples thereof. According to one or more embodiments described herein, the selection of the healthy model is based on identifying a landmark in the medical image of the subject and a corresponding landmark in the healthy model. A landmark can be any suitable internal or external feature, such as an organ (e.g., heart, liver, lung, etc.), a bone, and/or the like including combinations and/or multiples thereof.

According to one or more embodiments described herein, the selection of the healthy model is based on measurements of the subject and measurements for the healthy models. For example, a healthy model is selected by determining a plurality of measurements of the subject from the medical image of the subject. Such measurements can be, for example, a circumference of the head or torso of the subject, length, depth, and/or width of the head or torso of the subject, a size of an organ (e.g.,. heart, liver, lung, etc.) of the subject, and/or the like including combinations and/or multiples thereof. The selection is further performed by comparing the plurality of measurements of the subject to a plurality of measurements for each healthy model. For example, if measurements of the subject include length, width, and circumference of the torso of the subject, such measurements can be compared to corresponding measurements of healthy models. The healthy model is then selected to be representative of the subject based on the healthy model having measurements most similar to the measurements of the subject. According to one or more embodiments described herein, if the medical image of the subject is of a torso of the subject, the plurality of measurements of the subject may include at least five measurements of the subject. It should be appreciated that other numbers of measurements can be used (e.g., more than five or less than five). According to one or more embodiments described herein, the healthy model is selected based on a location of an organ of the subject. That is, a location of an organ (e.g., heart, liver, lung, etc.) of the subject is compared to a corresponding to location of the same organ in the healthy models, and a healthy model is selected based on the location of the organ being similar between the subject and the heathy model.

According to one or more embodiments described herein, the healthy models are generated according to the method 120 of FIG. 1B, which is now described in more detail. In particular, FIG. 1B depicts an example method 120 for generating healthy models according to an embodiment. Certain steps of the method 120 are described as computer-implemented steps. The computer may be, for example, any device comprising one or more processors and memory accessible by the one or more processors, the memory storing instructions that when executed by the one or more processors cause the computer to perform the relevant steps of the method 120. The method 120 may be implemented by any suitable system or apparatus, such as the apparatus 900 of FIG. 9. While an order of operations is indicated in FIG. 1B for illustrative purposes, the timing and ordering of such operations may vary where appropriate without negating the purpose and advantages of the examples set forth in detail herein.

At step 122, the method 120 includes receiving training data for a plurality of healthy subjects. The training data includes information about the healthy subjects. For example, the training data can include medical history information (e.g., past diagnoses, past treatments), demographic information (e.g., height, weight, gender, body mass), medical images (e.g., MRI images, CT images), and/or the like including combinations and/or multiples thereof.

At step 124, the method 120 includes analyzing the training data to identify commonalities among the plurality of healthy subjects. Examples of commonalities include height, weight, gender, body mass, and/or the like including combinations and/or multiples thereof. According to one or more embodiments described herein, analyzing the training data includes performing a principal component analysis (PCA) to identify the commonalities among the plurality of healthy subjects. In other embodiments, other approaches to performing the commonality analysis can be performed.

At step 126, the method 120 includes clustering the plurality of healthy subjects into clusters based at least in part on the commonalities among the plurality of healthy subjects. For example, subjects that are similar in height, weight, gender, and body mass may be clustered into a cluster. According to one or more embodiments described herein, the clustering is performed using k-means clustering. In other embodiments, other approaches to cluster can be performed.

At step 128, the method 120 includes generating the plurality of healthy models. Generating the healthy models includes, for each cluster, generating one of the plurality of healthy models based at least in part on the training data for the plurality of healthy subjects that are within the cluster. According to an embodiment, a model of one of the plurality of healthy subjects within the cluster is selected to be the healthy model. According to another embodiment, the healthy model for the cluster is generated using information from two or more of the healthy subjects within the cluster. According to one or more embodiments described herein, each of the healthy models is defined by a vector having a number of variables. The healthy model can be created from the variables of the healthy models of the healthy subjects within the cluster. As an example, the healthy model is an average of the variables of healthy models within the cluster.

According to one or more embodiments described herein, generating the plurality of healthy models includes selecting, for each cluster, a model of one of the healthy subjects within the cluster to be the healthy model. According to one or more embodiments described herein, generating the plurality of healthy models includes generating, for each cluster, the healthy model using information about at least two of the healthy subjects within the cluster. For example, the healthy model can be generated by averaging values from two or more healthy subjects within the cluster. According to one or more embodiments described herein, each of the healthy subjects has a medical image associated therewith, and measurements for each healthy subject are extracted from the medical image associated with each healthy subject. The measurements are used to select a healthy model as described with reference to step 104, for example.

With continued reference to FIG. 1A, at step 106, the method 100 includes receiving a selection of locations on the healthy model to place transducers to treat the abnormality. According to one or more embodiments described herein, the selection of locations on the healthy model to place the transducers to treat the abnormality is based on the location of the abnormality with respect to the subject. As an example, if the abnormality is in the head of the subject, one or more of locations on the healthy model may include a first pair of transducers with a front-back relationship and a second pair of transducers with a left-right relationship (such as the transducer layout in FIG. 8). As an example, if the abnormality is in the torso of the subject, one or more of locations on the healthy model may include a first pair of transducers with a front-back relationship and a second pair of transducers with a left-right relationship. According to one or more embodiments described herein, the selection of locations on the healthy model to place the transducers to treat the abnormality is based at least in part on a preselected list of locations based on, for example, the healthy model and a general location of an abnormality in the subject (e.g., in the center of the head, in the front of the head, in the front left of the head, in the upper torso, in the upper left torso, etc.). The locations on the healthy model can be selected automatically (e.g., based on analyzing one or more medical images of the subject), manually (e.g., a healthcare provider can select the locations), and/or the like including combinations and/or multiples thereof.

At step 108, the method 100 includes receiving an indication of a region of interest (ROI) in the healthy model. According to one or more embodiments described herein, the region of interest is defined by a gross tumor volume (GTV) for the abnormality, which is then used to calculate the dosage of treatment (at step 112). The indication of the ROI can be determined automatically (e.g., based on analyzing one or more medical images of the subject to determine the ROI), manually (e.g., a healthcare provider can indicate the ROI), and/or the like including combinations and/or multiples thereof.

At step 110, the method 100 includes calculating, by at least one processor (e.g., the one or more processors 902 of FIG. 9), for each of the plurality of locations (from step 106), a dosage of TTFields treatment for the region of interest in the healthy model without modifying the healthy model to include abnormal tissue. According to one or more embodiments described herein, the healthy model defines healthy tissue having an electrical property, and calculating the dosage of TTFields treatment is based at least in part on the electrical property. For example, the healthy model can define a conductivity of one or more types of healthy tissue, and the dosage of TTFields treatment may be calculated using the conductivity for each of the one or more types of healthy tissue. Calculating the dosage of TTFields treatment is further described in more detail in U.S. Patent Application Publication No. 2020/0023179, entitled “USING POWER LOSS DENSITY AND RELATED MEASURES TO QUANTIFY THE DOSE OF TUMOR TREATING FIELDS (TTFIELDS)” and U.S. Patent Application Publication No. 2021/0196943, entitled “METHODS, SYSTEMS, AND APPARATUSES FOR FAST APPROXIMATION OF ELECTRIC FIELD DISTRIBUTION,” the entire contents of both of which are incorporated by reference herein. It should be appreciated that these calculations in step 110 involve solving complex algorithms using large data sets associated with the healthy model and associated with the subject and, as such, require the use of a computer apparatus, as the human mind is not capable of performing the required calculations.

At step 112, the method 100 includes selecting one or more of the locations as recommended locations based on the dosage calculated in step 110. According to one or more embodiments described herein, the location with a highest dosage for the ROI may be selected as a recommended location. According to one or more embodiments described herein, the location with a second highest dosage for the ROI may be selected as a recommended location. According to one or more embodiments described herein, the location with a highest dosage for the ROI that also matches other criteria may be selected as a recommended location. As an example, other criteria to be considered may include: avoiding one or more avoidance areas of the subject (e.g., a nipple, a surgical scar, an eye, an ear, a mouth, a nose, irritated skin, a sensitive area, etc.); and providing maximized patient comfort. The recommended locations can be selected automatically (e.g., based on analyzing the dosages and the locations), manually (e.g., a healthcare provider can analyze the dosages and the locations), and/or the like including combinations and/or multiples thereof.

At step 114, the method 100 includes providing the one or more recommended locations selected in step 112. According to one or more embodiments described herein, each recommended location may be output as a representation of one or more pairs of transducers and locations for the one or more pairs of transducers on the subject. As an example, the representation of the subject may be based on the healthy model or the subject. As an example, a display is used to show a representation of the transducers and/or the locations on the subject. As an example, the output of the representation may be in form of a document.

At step 116, the method 100 includes receiving a selection of a recommended location. The recommended location can be selected automatically (e.g., based on analyzing one or more medical images of the subject), manually (e.g., a healthcare provider can select the recommended location from the possible locations), and/or the like including combinations and/or multiples thereof. The selected location can then be used to provide the dosage of TTFields treatment to the subject.

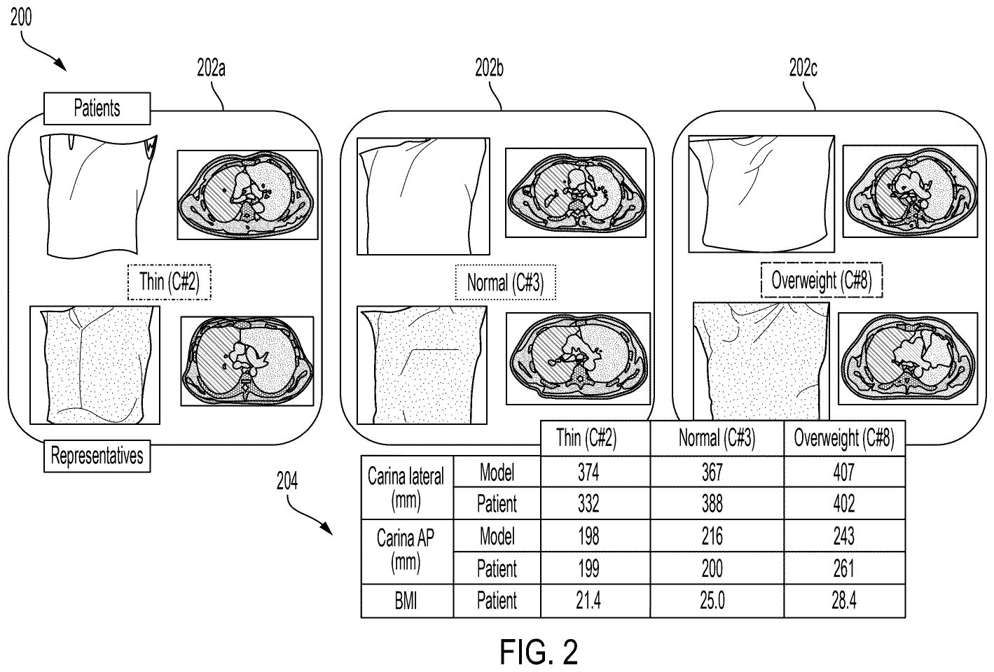

FIG. 2 depicts examples of healthy models 202a-202c. In this example, the healthy model 202a is a healthy model for a “thin” classification, the healthy model 202b is a healthy model for a “normal” classification, and the healthy model 202c is a healthy model for an “overweight” classification. As shown in the table 204, various values are associated with each of the healthy models 202a-202c, such as values for a carina lateral measurement, a carina AP measurement, and a body mass index. It should be appreciated that additional and/or alternative values can be associated with one or more of the healthy models 202a-202c in other embodiments. As can be seen in the table 204, corresponding values for the subject (e.g., “Patient”) are also shown.

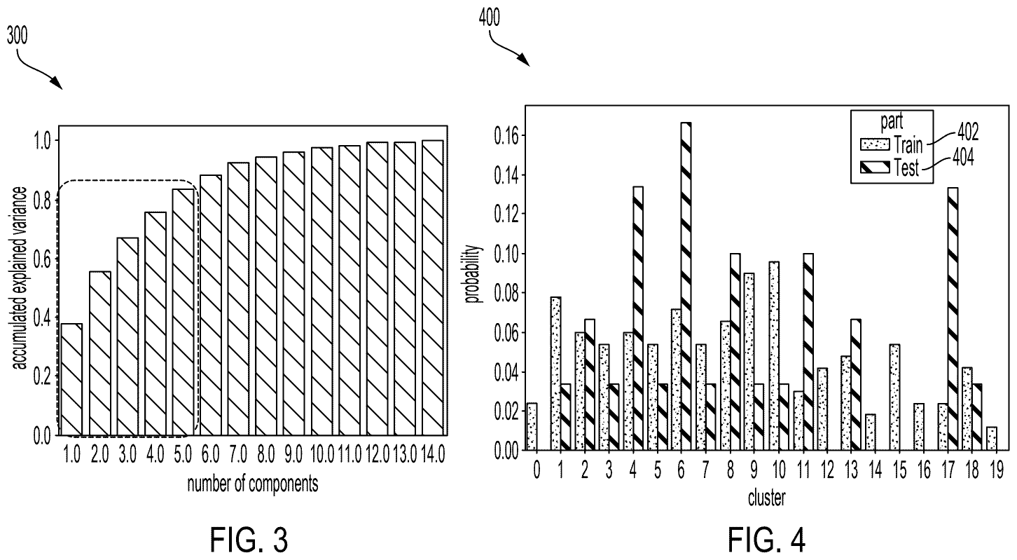

FIG. 3 depicts an example plot 300 used for clustering healthy models. In this example, a number of components are identified and plotted based on their accumulated explained variance. The components can be identified automatically (e.g., by a machine learning model trained to identify components from data of subjects), manually (e.g., selected by a subject matter expert), and/or combinations thereof. Non-limiting examples of components are: a carina anteroposterior (AP) measurement, a carina lateral measurement, a carina thoracic cavity lateral measurement a carina thoracic cavity lateral AP measurement, a carina mediastinum width measurement, a top liver AP measurement, a top liver lateral measurement, a top liver thoracic cavity lateral measurement, a top liver thoracic cavity AP measurement, a suprasternal notch AP measurement, a suprasternal notch lateral measurement, a suprasternal notch thoracic cavity lateral measurement, a suprasternal notch thoracic cavity AP measurement, a suprasternal notch to top liver measurement, and/or the like including combinations and/or multiples thereof. Principal component analysis can be used to identify a certain number of features as being relevant. For example, although fourteen components are used in the example of FIG. 3, it can be shown that the first five components account for 80% of the data and thus the remaining components can be ignored or discarded according to one or more embodiments described herein. In such cases, each healthy model would have a vector of five variables, each of the five variables corresponding to one of the five components.

FIG. 4 depicts an example plot 400 used for clustering healthy models. In this example, training data 402 and test data 404 are plotted for each of twenty clusters based on their respective probabilities. The training data 402 represents data about subjects that are used to train a machine learning model, for example, to perform clustering, and the test data 404 represents data about subjects used to test or verify the machine learning model after training. It should be appreciated that the training data 402 and the testing data 404 are different data according to one or more embodiments described herein.

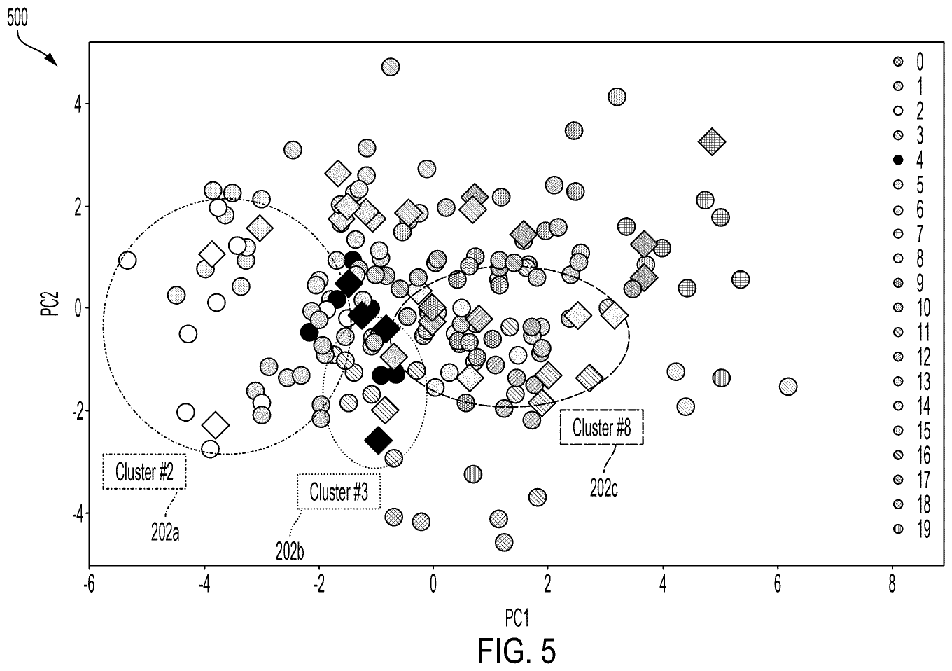

FIG. 5 depicts an example plot 500 of clusters of healthy models. In this example, data points for twenty different clusters are shown, corresponding to the plot 400. Three clusters 502a, 502b, 502c are shown, which correspond to the heathy models 202a, 202b, 202c, respectively, of FIG. 2. In the example of FIG. 5, data points for each cluster fall within a geometric shape, such as a circle or ellipse, as shown. According to one or more embodiments described herein, a centroid of the shape can be determined and can be used as the healthy model for that cluster. According to one or more embodiments described herein, one of the data points within the cluster can be selected to be the healthy model of the cluster.

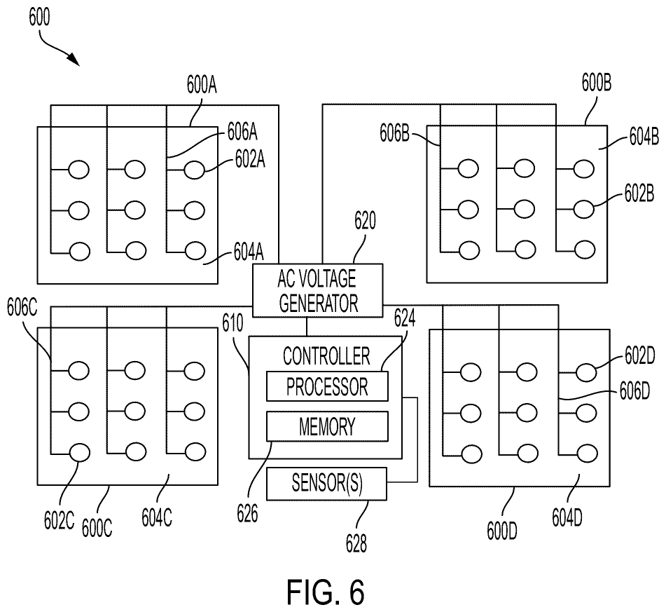

FIG. 6 depicts an example apparatus 600 to apply alternating electric fields (e.g., TTFields) to the subject's body. The system may be used for treating a target region of a subject's body with an alternating electric field. According to one or more embodiments described herein, at least two transducers are used to provide alternating electric fields, such as TTFields, to a subject. For example, one electrode element or multiple electrode elements can be used to provide the alternating electric fields, such as TTFields, to the subject. According to one or more embodiments described herein, one or more pairs of transducers are used to deliver alternating electric fields, such as TTFields, to the subject. In an example, the target region may be in the subject's brain, and an alternating electric field may be delivered to the subject's body via transducers (e.g., a pair of transducers, two pairs of transducer arrays, and/or the like including combinations and/or multiples thereof) positioned on a head of the subject's body (such as, for example, in FIG. 8, which has four transducers 800). In another example, the target region may be in the subject's torso, and an alternating electric field may be delivered to the subject's body via transducers (e.g., a pair of transducers, two pairs of transducer arrays, and/or the like including combinations and/or multiples thereof) positioned on at least one of a thorax, an abdomen, or one or both thighs of the subject's body. Other transducer array placements on the subject's body may be possible.

The example apparatus 600 depicts an example system having four transducers (or “transducer arrays”) 600A-D. It should be appreciated that other numbers of transducers can be used in other embodiments, such as two transducers or more than four transducers. Each transducer 600A-D may include substantially flat electrode elements 602A-D positioned on a substrate 604A-D and electrically and physically connected (e.g., through conductive wiring 206A-D). The substrates 604A-D may include, for example, cloth, foam, flexible plastic, and/or conductive medical gel. Two transducers (e.g., 600A and 600D) may be a first pair of transducers configured to apply an alternating electric field to a target region of the subject's body. The other two transducers (e.g., 600B and 600C) may be a second pair of transducers configured to similarly apply an alternating electric field to the target region.

The transducers 600A-D may be coupled to an AC voltage generator 620, and the system may further include a controller 610 communicatively coupled to the AC voltage generator 620. The controller 610 may include a computer having one or more processors 624 and memory 626 accessible by the one or more processors. The memory 626 may store instructions that when executed by the one or more processors control the AC voltage generator 620 to induce alternating electric fields between pairs of the transducers 600A-D according to one or more voltage waveforms and/or cause the computer to perform one or more methods disclosed herein. The controller 610 may monitor operations performed by the AC voltage generator 620 (e.g., via the processor(s) 624). One or more sensor(s) 628 may be coupled to the controller 610 for providing measurement values or other information to the controller 610.

In some embodiments, the voltage generation components may supply the transducers 600A-D with an electrical signal having an alternating current waveform at frequencies in a range from about 50 kHz to about 1 MHz and appropriate to deliver TTFields treatment to the subject's body

The electrode elements 602A-D may be capacitively coupled. In one example, the electrode elements 602A-D are ceramic electrode elements coupled to each other via conductive wiring 606A-D. When viewed in a direction perpendicular to its face, the ceramic electrode elements may be circular shaped or non-circular shaped. In other embodiments, the array of electrode elements are not capacitively coupled, and there is no dielectric material (such as ceramic, or high dielectric polymer layer) associated with the electrode elements.

The structure of the transducers 600A-D may take many forms. The transducers may be affixed to the subject's body or attached to or incorporated in clothing covering the subject's body. The transducer may include suitable materials for attaching the transducer to the subject's body. For example, the suitable materials may include cloth, foam, flexible plastic, and/or a conductive medical gel. The transducer may be conductive or non-conductive.

The transducer may include any desired number of electrode elements. Various shapes, sizes, and materials may be used for the electrode elements. Any constructions for implementing the transducer (or electric field generating device) for use with embodiments of the invention may be used as long as they are capable of (a) delivering TTFields to the subject's body and (b) being positioned at the locations specified herein. In some embodiments, at least one electrode element of the first, the second, the third, or the fourth transducer may include at least one ceramic disk that is adapted to generate an alternating electric field. In some embodiments, at least one electrode element of the first, the second, the third, or the fourth transducer may include a polymer film that is adapted to generate an alternating electric field.

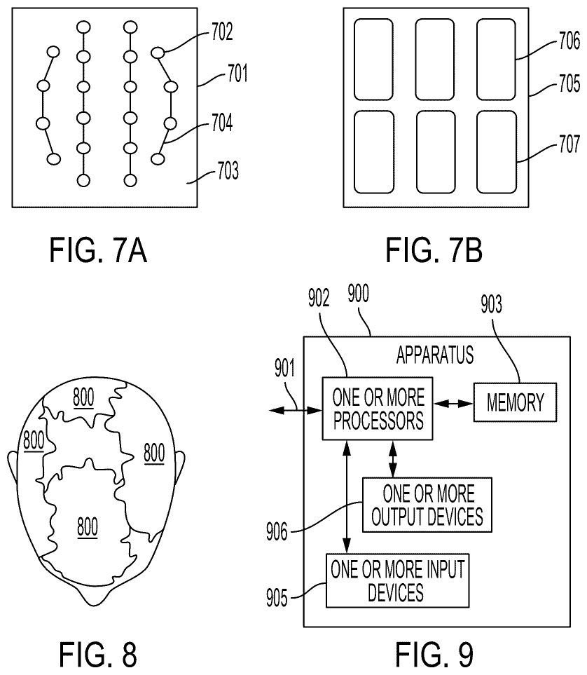

FIG. 7A illustrates a schematic view of an exemplary design of a transducer for applying alternating electric fields. Transducer 701 includes twenty electrode elements 702, which are positioned on substrate 703, and electrode elements 702 are electrically and physically connected to one another through a conductive wiring 704. In some embodiments, electrode elements 702 may include a ceramic disk.

FIG. 7B illustrates a schematic view of an exemplary design of a transducer for applying alternating electric fields. Transducer 705 may include substantially flat electrode elements 706. In some embodiments, electrode elements 706 are non-ceramic dielectric materials positioned over flat conductors. Examples of non-ceramic dielectric materials positioned over flat conductors may include polymer films disposed over pads on a printed circuit board or over substantially planar pieces of metal. In some embodiments, such polymer films have a high dielectric constant, such as, for example, a dielectric constant greater than 10. In some embodiments, electrode elements 706 may have various shapes. For example, the electrode elements may be triangular, rectangular, circular, oval, ovaloid, ovoid, or elliptical in shape or substantially triangular, substantially rectangular, substantially circular, substantially oval, substantially ovaloid, substantially ovoid, or substantially elliptical in shape. In some embodiments, each of electrode elements 706 may have a same shape, similar shapes, and/or different shapes.

FIG. 9 depicts an example computer apparatus for use with one or more embodiments described herein. As an example, apparatus 900 may be a computer to implement certain inventive techniques disclosed herein, such as determining transducer locations for delivery of TTFields based on models of healthy subjects. As an example, method 100 of FIG. 1A and/or the method 120 of FIG. 1B may be performed by a computer, such as the apparatus 900. As an example, steps 102 to 116 of FIG. 1A and/or the steps 122 to 128 of FIG. 1B may be performed by a computer, such as apparatus 900. In some embodiments, controller 610 of FIG. 6 may be implemented with apparatus 900. As an example, some or all of the steps in the method illustrated in FIGS. 1A and 1B may be performed on a single apparatus 900. As an example, the steps in the method illustrated in FIGS. 1A and 1B may be performed by one, two, three, four, or more apparatuses 900. As an example, the apparatus 900 may be a portable computer device (e.g., a mobile device, a tablet or a laptop).

The apparatus 900 may include one or more processors 902, memory 903, one or more input devices 905, and one or more output devices 906.

Input to the apparatus 900 may be provided by one or more input devices 905, provided from one or more input devices in communication with the apparatus 900 via link 901 (e.g., a wired link or a wireless link; e.g., with a direct connection or over a network), and/or provided from another computer(s) in communication with the apparatus 900 via link 901.

Output for the apparatus 900 may be provided by one or more output devices 906, provided to one or more output devices in communication with the apparatus 900 via link 901, and/or provided from another computer(s) in communication with the apparatus 900 via link 901. The one or more output devices 906 may provide the status of the operation according to some embodiments described herein, such as transducer array selection, voltages being generated, and other operational information. The output device(s) 906 may provide visualization data according to some embodiments described herein. The one or more output devices 906 may include one more displays and one or more speakers. The output device(s) 906 may display healthy models, a model of the abnormality, and/or recommended locations as described with respect to FIGS. 1A and 1B, and/or another suitable outputs associated with determining transducer locations for delivery of tumor treating fields based on models of healthy subjects according to one or more embodiments described herein.

In some embodiments, one or more input devices 905 and one or more output devices 906 may be combined into one or more unitary input/output devices (e.g., a touch screen).

In some embodiments, based on input from one or more input devices 905 or input from outside the apparatus 900 via the link 901, the one or more processors 902 may perform operations as described herein. For example, based on input 901, the one or more processors 902 may generate control signals to control the voltage generator to implement one or more embodiments described herein. As an example, user input may be received from the one or more input devices 905. As an example, input may be from another computer in communication with the apparatus 900 via link 901. As an example, input may be from one or more input devices in communication with the apparatus 900 via link 901.

In some embodiments, the one or more processors 902 may perform operations as described herein and provide results of the operations as output. As an example, output may be provided to the one or more output devices 906. As an example, output may be provided to another computer in communication with the apparatus 900 via link 901. As an example, output may be provided to one or more output devices in communication with the apparatus 900 via link 901.

The memory 903 may be accessible by the one or more processors 902 so that the one or more processors 902 may read information from and write information to the memory 903. The memory 903 may store instructions that, when executed by the one or more processors 902, implement one or more embodiments described herein. The memory 903 may be a non-transitory computer readable medium (or a non-transitory processor readable medium) containing a set of instructions thereon for determining transducer locations for delivery of tumor treating fields based on models of healthy subjects, wherein when executed by a processor (such as one or more processors 902), the instructions cause the processor to perform one or more methods discussed herein. As an example, the apparatus 900 may be a computer, and memory of the computer may store an app, a program, or a software to perform embodiments described herein.

The apparatus 900 may be an apparatus for determining transducer locations for delivery of tumor treating fields based on models of healthy subjects, the apparatus including: one or more processors (such as one or more processors 902); and memory (such as memory 903) accessible by the one or more processors, the memory storing instructions that when executed by the one or more processors, cause the apparatus to perform one or more methods described herein.

The memory 903 may be a non-transitory processor readable medium containing a set of instructions thereon for determining transducer locations for delivery of tumor treating fields based on models of healthy subjects, wherein when executed by one or more processors (such as one or more processors 902), the instructions cause the one or more processors to perform one or more methods described herein.

### Illustrative Embodiments

The invention includes other illustrative embodiments (“Embodiments”) as follows.

Embodiment 1. A computer-implemented method for determining transducer locations for delivery of tumor treating fields based on models of healthy subjects, the method comprising: receiving a medical image of a subject having an abnormality; receiving a selection of a healthy model from a plurality of healthy models, the healthy model being representative of the subject, the selection based on the medical image of the subject; receiving a selection of locations on the healthy model to place transducers to treat the abnormality of the subject without identifying a location of the abnormality in the healthy model; receiving an indication of a region of interest in the healthy model; and calculating, by at least one processor, for each of the locations, a dosage of tumor treating fields treatment in the region of interest of the healthy model without modifying the healthy model to include abnormal tissue.

Embodiment 2. The method of embodiment 1, wherein the selection of the healthy model is based on identifying a landmark in the medical image of the subject and a corresponding landmark in the healthy model.

Embodiment 3. The method of embodiment 1, wherein the selection of the healthy model comprises.

Embodiment 3A. The method of embodiment 3, wherein the plurality of measurements of the subject comprise at least one of a circumference of an aspect of the subject, a length of an aspect of the subject, a width of the aspect of the subject, a depth of an aspect of the subject, and a size of an organ of the subject.

Embodiment 3B. The method of embodiment 3, wherein the medical image of the subject is of a torso of the subject, and wherein the plurality of measurements of the subject comprise at least 5 measurements of the subject.

Embodiment 4. The method of embodiment 1, wherein the selection of the healthy model is further based on a location of an organ of the subject.

Embodiment 5. The method of embodiment 1, wherein the selection of the locations on the healthy model to place transducers to treat the abnormality is based at least in part on conductivities for at least one tissue type included in the healthy model.

Embodiment 6. The method of embodiment 5, wherein calculating the dosage of tumor treating fields treatment is based at least in part on the conductivities for the at least one tissue type included in the healthy model.

Embodiment 7. The method of embodiment 1, wherein the healthy model defines healthy tissue having an electrical property, wherein calculating the dosage of treatment is based at least in part on the electrical property.

Embodiment 8. The method of embodiment 1, wherein each of the plurality of healthy models is segmented based on tissue type.

Embodiment 9. The method of embodiment 1, wherein each of the plurality of healthy models is representative of a group of the healthy subjects without abnormalities.

Embodiment 10. The method of embodiment 1, wherein the plurality of healthy models is based on the healthy subjects clustered into groups.

Embodiment 11. The method of embodiment 1, wherein the plurality of healthy models are generated by: receiving training data for the healthy subjects; analyzing the training data to identify commonalities among the healthy subjects; clustering the healthy subjects into clusters based at least in part on the commonalities among the healthy subjects; and generating the plurality of healthy models, wherein the generating comprises, for each cluster, generating one of the plurality of healthy models based at least in part on the training data for the healthy subjects that are within the cluster.

Embodiment 12. The method of embodiment 11, wherein generating the plurality of healthy models comprises selecting, for each cluster, a model of one of the healthy subjects within the cluster to be the healthy model.

Embodiment 13. The method of embodiment 11, wherein generating the plurality of healthy models comprises generating, for each cluster, the healthy model using information about at least two of the healthy subjects within the cluster.

Embodiment 14. The method of embodiment 11, wherein each of the healthy subjects has a medical image associated therewith, wherein measurements for each healthy subject are extracted from the medical image associated with each healthy subject

Embodiment 15. The method of embodiment 11, wherein analyzing the training data comprises performing a principal component analysis to identify the commonalities among the plurality of healthy subjects.

Embodiment 16. The method of embodiment 11, wherein the commonalities are based on principal components defined for each of the healthy subjects.

Embodiment 16A. The method of embodiment 16, wherein the principal components are defined by a subject matter expert.

Embodiment 16B. The method of embodiment 16, wherein the principal components are defined automatically based on analyzing the training data.

Embodiment 17. The method of embodiment 16, wherein the clustering is performed using k-means clustering.

Embodiment 17A. The method of embodiment 1, wherein each of the healthy models is defined by a vector having a number of variables.

Embodiment 18. The method of embodiment 1, wherein the medical image is at least one of a computed tomography (CT) image, a magnetic resonance imaging (MRI) medical image, or a positron emission tomography (PET) medical image.

Embodiment 18A. The method of embodiment 1, wherein the abnormality is a tumor.

Embodiment 18B. The method of embodiment 1, further comprising: selecting one or more of the locations as recommended locations based on the dosage; and providing the one or more recommended locations.

Embodiment 19. An apparatus for determining transducer locations for delivery of tumor treating fields based on models of healthy subjects, the apparatus comprising: one or more processors; and a memory accessible by the one or more processors, the memory storing instructions that when executed by the one or more processors, cause the apparatus to: receive a medical image of a subject having an abnormality; receive a selection of a healthy model from a plurality of healthy models, the healthy model being representative of the subject, the selection based on the medical image of the subject; receive a selection of locations on the healthy model to place transducers to treat the tumor of the subject without identifying a location of the abnormality in the healthy model; receive an indication of a region of interest in the healthy model; and calculate a dosage of tumor treating fields treatment in the region of interest of the healthy model without modifying the healthy model to include abnormal tissue associated with the abnormality.

Embodiment 19A. The apparatus of embodiment 19, wherein the plurality of healthy models are generated by: receiving training data for the healthy subjects; analyzing the training data to identify commonalities among the healthy subjects; clustering the healthy subjects into clusters based at least in part on the commonalities among the healthy subjects; and generating the plurality of healthy models, wherein the generating comprises, for each cluster, generating one of the plurality of healthy models based at least in part on the training data for the healthy subjects that are within the cluster.

Embodiment 19B. The apparatus of embodiment 19A, wherein generating the plurality of healthy models comprises selecting, for each cluster, a model of one of the healthy subjects within the cluster to be the healthy model.

Embodiment 19C. The apparatus of embodiment 19A, wherein generating the plurality of healthy models comprises generating, for each cluster, the healthy model using information about at least two of the healthy subjects within the cluster.

Embodiment 19D. The apparatus of embodiment 19A, wherein analyzing the training data comprises performing a principal component analysis to identify the commonalities among the healthy subjects.

Embodiment 20. A non-transitory processor readable medium containing a set of instructions thereon for determining transducer locations for delivery of tumor treating fields based on models of healthy subjects, wherein when executed by a processor, the instructions cause the processor to: receive a medical image of a subject having an abnormality; receive a selection of a healthy model from a plurality of healthy models, the healthy model being representative of the subject, the selection based on the medical image of the subject; receive a selection of locations on the healthy model to place transducers to treat the tumor of the subject without identifying a location of the abnormality in the healthy model; receive an indication of a region of interest in the healthy model; and calculate a dosage of tumor treating fields treatment in the region of interest of the healthy model without modifying the healthy model to include abnormal tissue associated with the abnormality.

Embodiment 21. A method, machine, manufacture, and/or system substantially as shown and described.

Optionally, for each embodiment described herein, the voltage generation components supply the transducers with an electrical signal having an alternating current waveform at frequencies in a range from about 50 kHz to about 1 MHz and appropriate to deliver TTFields treatment to the subject's body.

Embodiments illustrated under any heading or in any portion of the disclosure may be combined with embodiments illustrated under the same or any other heading or other portion of the disclosure unless otherwise indicated herein or otherwise clearly contradicted by context. For example, and without limitation, embodiments described in dependent claim format for a given embodiment (e.g., the given embodiment described in independent claim format) may be combined with other embodiments (described in independent claim format or dependent claim format).

Numerous modifications, alterations, and changes to the described embodiments are possible without departing from the scope of the present invention defined in the claims. It is intended that the present invention need not be limited to the described embodiments, but that it has the full scope defined by the language of the following claims, and equivalents thereof.

What is claimed is:

1. A computer-implemented method for determining transducer locations for delivery of tumor treating fields based on models of healthy subjects, the method comprising:
receiving a medical image of a subject having an abnormality;
receiving a selection of a healthy model from a plurality of healthy models, the healthy model being representative of the subject, the selection based on the medical image of the subject;
receiving a selection of locations on the healthy model to place transducers to treat the abnormality of the subject without identifying a location of the abnormality in the healthy model;
receiving an indication of a region of interest in the healthy model; and
calculating, by at least one processor, for each of the locations, a dosage of tumor treating fields treatment in the region of interest of the healthy model without modifying the healthy model to include abnormal tissue.

2. The method of claim 1, wherein the selection of the healthy model is based on identifying a landmark in the medical image of the subject and a corresponding landmark in the healthy model.

3. The method of claim 1, wherein the selection of the healthy model comprises:
determining a plurality of measurements of the subject from the medical image of the subject;
comparing the plurality of measurements of the subject to a plurality of measurements for each health model; and
selecting the healthy model being representative of the subject as the healthy model having measurements most similar to measurements of the subject.

4. The method of claim 3, wherein the selection of the healthy model is further based on a location of an organ of the subject.

5. The method of claim 1, wherein the selection of the locations on the healthy model to place transducers to treat the abnormality is based at least in part on conductivities for at least one tissue type included in the healthy model.

6. The method of claim 5, wherein calculating the dosage of tumor treating fields treatment is based at least in part on the conductivities for the at least one tissue type included in the healthy model.

7. The method of claim 1, wherein the healthy model defines healthy tissue having an electrical property, wherein calculating the dosage of treatment is based at least in part on the electrical property.

8. The method of claim 1, wherein each of the plurality of healthy models is segmented based on tissue type.

9. The method of claim 1, wherein each of the plurality of healthy models is representative of a group of the healthy subjects without abnormalities.

10. The method of claim 1, wherein the plurality of healthy models is based on the healthy subjects clustered into groups.

11. The method of claim 1, wherein the plurality of healthy models are generated by:
receiving training data for the healthy subjects;
analyzing the training data to identify commonalities among the healthy subjects;
clustering the healthy subjects into clusters based at least in part on the commonalities among the healthy subjects; and
generating the plurality of healthy models, wherein the generating comprises, for each cluster, generating one of the plurality of healthy models based at least in part on the training data for the healthy subjects that are within the cluster.

12. The method of claim 11, wherein generating the plurality of healthy models comprises selecting, for each cluster, a model of one of the healthy subjects within the cluster to be the healthy model.

13. The method of claim 11, wherein generating the plurality of healthy models comprises generating, for each cluster, the healthy model using information about at least two of the healthy subjects within the cluster.

14. The method of claim 11, wherein each of the healthy subjects has a medical image associated therewith, wherein measurements for each healthy subject are extracted from the medical image associated with each healthy subject.

15. The method of claim 11, wherein analyzing the training data comprises performing a principal component analysis to identify the commonalities among the plurality of healthy subjects.

16. The method of claim 11, wherein the commonalities are based on principal components defined for each of the healthy subjects.

17. The method of claim 16, wherein the clustering is performed using k-means clustering.

18. The method of claim 1, wherein the medical image is at least one of a computed tomography (CT) image, a magnetic resonance imaging (MRI) medical image, or a positron emission tomography (PET) medical image.

19. An apparatus for determining transducer locations for delivery of tumor treating fields based on models of healthy subjects, the apparatus comprising:
one or more processors; and
a memory accessible by the one or more processors, the memory storing instructions that when executed by the one or more processors, cause the apparatus to:
receive a medical image of a subject having an abnormality;
receive a selection of a healthy model from a plurality of healthy models, the healthy model being representative of the subject, the selection based on the medical image of the subject;
receive a selection of locations on the healthy model to place transducers to treat the tumor of the subject without identifying a location of the abnormality in the healthy model;
receive an indication of a region of interest in the healthy model; and
calculate a dosage of tumor treating fields treatment in the region of interest of the healthy model without modifying the healthy model to include abnormal tissue associated with the abnormality.

20. A non-transitory processor readable medium containing a set of instructions thereon for determining transducer locations for delivery of tumor treating fields based on models of healthy subjects, wherein when executed by a processor, the instructions cause the processor to:
receive a medical image of a subject having an abnormality;
receive a selection of a healthy model from a plurality of healthy models, the healthy model being representative of the subject, the selection based on the medical image of the subject;
receive a selection of locations on the healthy model to place transducers to treat the tumor of the subject without identifying a location of the abnormality in the healthy model;
receive an indication of a region of interest in the healthy model; and
calculate a dosage of tumor treating fields treatment in the region of interest of the healthy model without modifying the healthy model to include abnormal tissue associated with the abnormality.
